(19) United States
(12) Patent Application Publication
(10) Pub. No.: US 2024/0093247 A1
(43) Pub. Date: Mar. 21, 2024

(54) GENETICALLY ENGINEERED STRAIN OF ZYMOMONAS MOBILIS AND USES THEREOF

(71) Applicant: Hubei University, Wuhan (CN)

(72) Inventors: Shihui YANG, Wuhan (CN); Binan GENG, Wuhan (CN); Qiaoning HE, Wuhan (CN); Jun DU, Wuhan (CN); Mian LI, Wuhan (CN)

(73) Assignee: Hubei University, Wuhan (CN)

(21) Appl. No.: 18/468,813

(22) Filed: Sep. 18, 2023

(30) Foreign Application Priority Data
Sep. 19, 2022 (CN) 2022111380206

(51) Int. Cl. C12P 7/10; C12N 9/22; C12N 15/11; C12N 15/74; C12N 15/90

(52) U.S. Cl. CPC C12P 7/10; C12N 9/22; C12N 15/11; C12N 15/74; C12N 15/902; C12N 2310/20; C12N 2800/101; C12N 2800/80

(57) ABSTRACT

A genetically engineered strain of Z. mobilis and uses thereof are provided. The genetically engineered strain named ZMNP is obtained by knockouts of four endogenous plasmids of pZM32, pZM32, pZM36, pZM33, and pZM39 from strain ZM4-Cas12a. ZMNP contains a reduced genome and has a series of excellent traits, such as high transformation efficiency, enhanced tolerance to inhibitors, improved capability to use secondary mother liquor and other excellent performance. ZMNP can be used as a chassis cell to construct diverse cell factories for biochemical production using different feedstocks especially the lignocellulosic biomass.

### CROSS-REFERENCE TO RELATED APPLICATIONS

This disclosure claims priority to Chinese Patent Application NO: 202211138020.6, filed with China Intellectual Property Office on Sep. 19, 2022, the entire contents of which are incorporated herein by reference.

### SEQUENCE LISTING

The sequence listing xml file submitted herewith, named “Conamen_geneticum_ipsum_Zymomonae_mobilis_eiusque_applicationis.xml”, created on Sep. 14, 2023, and having a file size of 37,888 bytes, is incorporated by reference herein.

### TECHNICAL FIELD

This disclosure relates to Zymomonas mobilis. Specifically, this disclosure relates to genetically engineered strain of Zymomonas mobilis and uses thereof.

### BACKGROUND

The statements herein provide background information relevant to the present disclosure only and do not necessarily constitute prior art.

As a sustainable development of the modern manufacturing mode, bio-industry not only effectively alleviates the global pressure such as environment pollution and shortage of resources and energy, but also is an important part of the bio-economy. And it is the main driving force for reducing the dependence on fossil fuels and resource consumption, creating employment opportunities and promoting sustainable development. The core to the development of the bio-industry is the establishment of excellent industrial strains with intellectual property. Currently, a variety of strains with excellent characteristics have been obtained through genetic engineering for biosynthesis of various biochemicals. Streamline optimization of the microbial genome of these strains is an important strategy for building excellent microbial cell factories, which includes large-scale deletion of non-essential coding and non-coding regions in the genome to obtain the “minimal genome”, and knockdown of redundant genes to reduce cellular energy consumption and to enable more energy for product production.

As a facultative anaerobic Gram-negative bacterium, Zymomonas mobilis (Z. mobilis) has many unique physiological and excellent industrial characteristics. It is the only microorganism known to utilize the 2-ketto-3-deoxyy-6-phosphogluconic acid (Entner-Doudoroff, ED) pathway under anaerobic conditions, and has excellent characteristics such as high sugar absorption rate, high ethanol yield and ethanol tolerance. In recent years, Z. mobilis has attracted much attentions because of its ability to synthesize platform biochemicals such as ethanol, lactic acid, 2-3 butanediol, isobutanol, and PHB. Compared with other model microorganisms Saccharomyces cerevisiae (12.12 Mb) and Escherichia coli (5.15 Mb), Z. mobilis (2.14 Mb) has a small genome to facilitate genome streamlining and optimization, which is an ideal industrial microbiological chassis cell.

### SUMMARY

In a first aspect, embodiments disclose a genetically engineered strain of Z. mobilis, named ZMNP. The ZMNP is obtained by knockouts of four endogenous plasmids, pZM32, pZM36, pZM33, and pZM39, a nuclease Cas12a gene and a spectinomycin gene from a strain named ZM4-Cas12a. The stain ZM4-Cas12a is a recombinant strain prepared by integrating the nuclease Cas12a gene, which is derived from F. novicida, and the spectinomycin gene into a ZMO0038 site on a genome of strain ZM4 by homologous recombination, and using an inducible promoter Ptet to control a expression of the nuclease Cas12a gene. Among these, a number in Genbank of pZM32 is No. CP023678; a number in Genbank of pZM33 is No. NZ_P023679; a number in Genbank of pZM36 is No. CP023680 and a number in Genbank of pZM39 is No. CP023681.

In a second aspect, embodiments disclose a method for preparing a genetically engineered strain of Z. mobilis, named ZMNP. The ZMNP is obtained by knockouts of four endogenous plasmids, pZM32, pZM36, pZM33, and pZM39, a nuclease Cas12a gene and a spectinomycin gene from a strain named ZM4-Cas12a. The stain ZM4-Cas12a is a recombinant strain prepared by integrating the nuclease Cas12a gene, which is derived from F. novicida, and the spectinomycin gene into a ZMO0038 site on a genome of strain ZM4 by homologous recombination, and using an inducible promoter Ptet to control a expression of the nuclease Cas12a gene. Among these, a number in Genbank of pZM32 is No. CP023678; a number in Genbank of pZM33 is No. NZ_P023679; a number in Genbank of pZM36 is No. CP023680 and a number in Genbank of pZM39 is No. CP023681.

And the method includes:

preparing a first editing plasmid used to targetedly knock out the pZM32 and the pZM36, a second editing plasmid used to targetedly knock out the pZM33, a third editing plasmid used to targetedly knock out the pZM39, a fourth editing plasmid used to replace a toxin-antitoxin operon of the pZM39, and a fifth editing plasmid used to targetedly knock out a nuclease Cas12a gene and a spectinomycin gene with gene ZMO0038;

transferring the first editing plasmid into the ZM4-Cas12a to obtain a strain named ZM4-Cas12aΔ32Δ36 whose pZM32 and pZM36 have been targetedly knocked out;

transferring the second editing plasmid into the strain ZM4-Cas12aΔ32Δ36 to obtain a strain named ZM4-Cas12aΔ32Δ33Δ36 whose the pZM32, pZM36 and pZM33 have been targetedly knocked out;

transferring the fourth editing plasmid into the ZM4-Cas12aΔ32Δ33Δ36 to obtain a strain named ZM4-Cas12aΔ32Δ33Δ36ΔTA::Cm that the toxin-antitoxin operon on pZM39 has been replaced by a chloramphenicol gene (Cm);

transferring the third editing plasmid into the strain ZM4-Cas12aΔ32Δ33Δ36ΔTA::Cm to obtain a genetically modified ZM4-Cas12a with four endogenous plasmids, pZM32, pZM36, pZM33, and pZM39 knocked out; and

transferring the fifth editing plasmid into the genetically modified ZM4-Cas12a to obtain the genetically engineered strain of ZMNP.

In a third aspect, embodiments disclose a use of the genetically engineered strain described in the first aspect or the genetically engineered strain obtained by the method described in the second aspect in the preparation of ethanol fermentation chassis bacteria.

In a fourth aspect, embodiments disclose a use of the genetically engineered strain described in the first aspect or the genetically engineered strain obtained by the construction method described in ethanol fermentation.

In a fifth aspect, embodiments disclose a use of the genetically engineered strain described in the first aspect or the genetically engineered strain obtained by the construction method described in the preparation of the chassis bacteria with a low ROS level.

### BRIEF DESCRIPTION OF DRAWINGS

FIG. 1A illustrates a flow chart of the preparation of the genetically engineered strain of Z. mobilis ZM4 (ZM4-Cas12aΔ32Δ33Δ36) in accordance with embodiments.

FIG. 1B illustrates a flow chart of the preparation of the genetically engineered strain of Z. mobilis ZM4 (ZMNP) in accordance with embodiments.

FIG. 2 illustrates the verification results by agarose gel electrophoresis of ZM4-Cas12a, ZM4-Cas12aΔ32Δ36, ZM4-Cas12aΔ32Δ33Δ36 and ZMNP(a), and results by agarose gel electrophoresis of the replenishment of ZMO0038 succeeded with the knockout of Cas12a and spectinomycin in ZMNP compared with the positive control strain ZM4(b). Lane “Out” represents a result of PCR with a pair of primers upstream and downstream of the ZMO0038 gene. Lane “in” represents a result of PCR with a primer of the ZMO0038 upstream gene and a primer inside from the ZMO0038 gene.

FIG. 3 illustrates diagrams of ZMNP and ZM4 by transmission electron microscope, in accordance with embodiments.

FIG. 4 illustrates a chart of results of transformation efficiency of ZM4 and ZMNP that have been calculated by colony number, in accordance with embodiments.

FIG. 5 illustrates a growth curve chart of ZMNP and ZM4 at 30° C.(a) and 40° C.(b), a glucose consumption chart and an ethanol generation metabolism chart of ZMNP and ZM4 at different times, in accordance with embodiments.

FIG. 6 illustrates a growth curve chart (a) of ZMNP and ZM4 in RMG5, and a ROS test result chart (b) of ZMNP and ZM4 in RMG5, in accordance with embodiments.

FIG. 7 illustrates a growth curve chart (a) of ZMNP and ZM4 in MMG5, and a ROS test result chart (b) of ZMNP and ZM4 in MMG5, in accordance with embodiments.

FIG. 8 illustrates a growth curve chart (a) of ZMNP and ZM4 in RMAce, and a ROS test result chart (b) of ZMNP and ZM4 in RMAce, in accordance with embodiments.

FIG. 9 illustrates a growth curve chart (a) of ZMNP and ZM4 in MMAce, and a ROS test result chart (b) of ZMNP and ZM4 in MMAce, in accordance with embodiments.

FIG. 10 illustrates a growth curve chart (a) of ZMNP and ZM4 in RMF, and a ROS test result chart (b) of ZMNP and ZM4 in RMF, in accordance with embodiments.

FIG. 11 illustrates a growth curve chart (a) of ZMNP and ZM4 in MMF, and a ROS test result chart (b) of ZMNP and ZM4(a) in MMF, in accordance with embodiments.

FIG. 12 illustrates a growth curve chart (a) of ZMNP and ZM4 in RMEth, and a ROS test result chart (b) of ZMNP and ZM4 in RMEth, in accordance with embodiments.

FIG. 13 illustrates a growth curve chart (a) of ZMNP and ZM4 in MMEth, and a ROS test result chart (b) of ZMNP and ZM4 in MMEth, in accordance with embodiments.

FIG. 14 illustrates a chart of glucose consumption and ethanol production of ZMNP and ZM4 in the secondary mother liquor of the cellulose hydrolysate of the corn cob, in accordance with embodiments.

FIG. 15 illustrates a flow chart of the preparation of the forth editing plasmid, in accordance with embodiments.

FIG. 16 illustrates a schematic diagram of gene editing of the replacement of the Cas12a and spectinomycin gene with gene ZMO0038 by the fifth editing plasmid, in accordance with embodiments.

FIG. 17 illustrates a structural chart of the plasmid of pEZ-HsdSp, in accordance with embodiments.

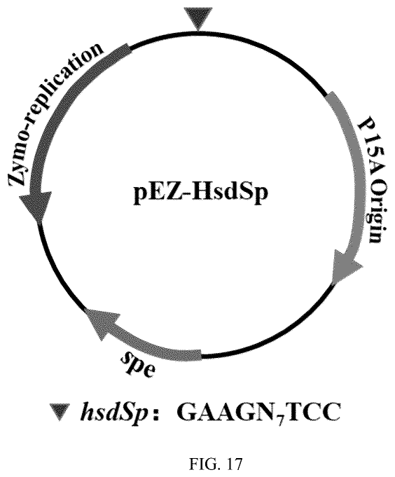

### DETAILED DESCRIPTION OF THE EMBODIMENTS

Through genome sequencing, the inventor of this disclosure has found that: the genome of ZM4 includes four endogenous plasmids: pZM32 (32,791 bp), pZM33 (33,006 bp), pZM36 (36,494 bp) and pZM39 (39,266 bp), these plasmids are about 6.43% of the total size of its genome, and the copy numbers of these four plasmids in ZM4 strains range 1 to 2 under anaerobic conditions, and 1 to 6 under aerobic conditions.

In this disclosure, ZM4-Cas12a is set as a original strain, and four endogenous plasmids are knocked out by means of genetic engineering to obtain a strain named ZMNP. That makes the ZMNP obtain a reduced genome, and a reduced energy consumption. And the means of genetic engineering also provided ZMNP a series of excellent traits, such as high transformation efficiency, more tolerance to inhibitors, better use of secondary mother liquor. These traits make ZMNP to be a tractable industrial microbial chassis cell.

In a first aspect, embodiments disclose a genetically engineered strain of Z. mobilis, named ZMNP. The ZMNP is obtained by knockouts of four endogenous plasmids, pZM32, pZM36, pZM33, pZM39, and a nuclease Cas12a gene and a spectinomycin gene from a strain named ZM4-Cas12a. Among these, the stain ZM4-Cas12a is a recombinant strain prepared by integrating the nuclease Cas12a gene derived from F. novicida with an inducible promoter Ptet, and the spectinomycin gene into the ZMO0038 site on the genome of ZM4 by homologous recombination. Thus, the recombinant strain ZM4-Cas12a contains a native Type I-F CRISPR-Cas gene editing system and an exogenous CRISPR-Cas12a editing system. A method of the recombinant preparation of the ZM4-Cas12a refers to “Establishment and application of a CRISPR-Cas12a assisted genome-edit system In Zymomonas mobilis[J], Microbial Cell Factories, 2019, 18:162”. Among these, the four endogenous plasmids in the ZM4-Cas12a strain are named in the following order by size and Genbank No: pZM32 (32,791 bp, Genbank No. CP023678), pZM33 (33,006 bp, Genbank No. NZ_P023679), pZM36 (36,494 bp, Genbank No. CP023680) and pZM39 (39,266 bp, Genbank No. CP023681).

The strain ZMNP has the following performances:
(1) ZMNP has a reduced genome. This can enhance the predictability and operability of its gene pathways, and makes ZMNP more tractable to be a chassis cell.
(2) ZMNP has a higher transformation efficiency than its parental strain ZM4 to facilitate the introduction of foreign DNA to function.
(3) ZMNP has an enhanced tolerance to inhibitors.
(4) ZMNP can use the glucose efficiently in a secondary mother liquor of the corncob hydrolysate, thus reduced the time and cost of ethanol production.

In a second aspect, embodiments disclose a method for preparing a genetically engineered strain of Z. mobilis. The described genetically engineered strain, named ZMNP, is obtained by eliminating the four endogenous plasmids, pZM32, pZM36, pZM33, pZM39, and replacing a nuclease Cas12a gene and a spectinomycin gene with gene ZMO0038.

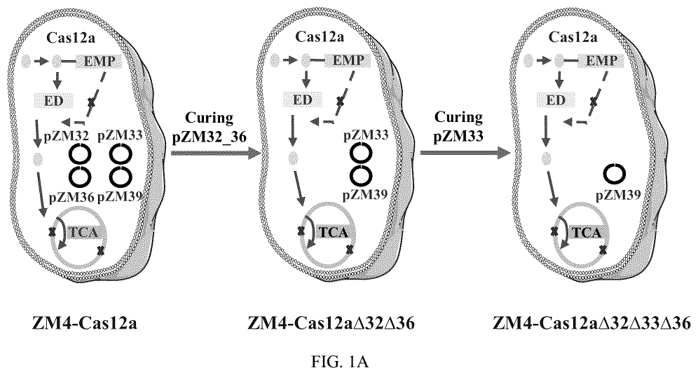

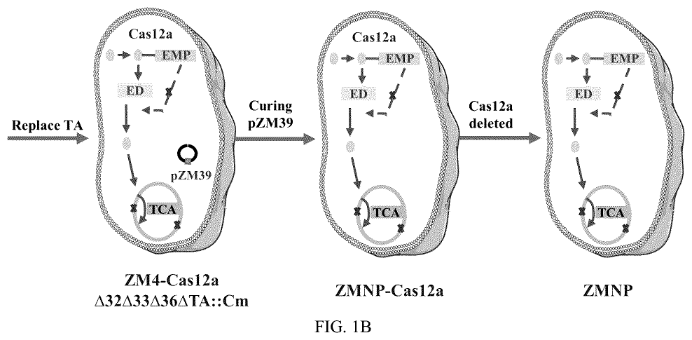

As shown in FIG. 1A and FIG. 1B, the method includes:

preparing a first editing plasmid used to targetedly knock out the pZM32 and the pZM36, a second editing plasmid used to targetedly knock out the pZM33, a third editing plasmid used to targetedly knock out the pZM39, a forth editing plasmid used to replace toxin-antitoxin system operon on pZM39, and a fifth editing plasmid used to targetedly knock out the Cas12a and the spectinomycin gene,

transferring the first editing plasmid into the strain ZM4-Cas12a to obtain a strain ZM4-Cas12aΔ32Δ36 that the pZM32 and pZM36 have been targetedly knocked out,

transferring the second editing plasmid into the strain ZM4-Cas12aΔ32Δ36 to obtain a strain named ZM4-Cas12aΔ32Δ33Δ36 that the pZM32, pZM36 and pZM33 have been targetedly knocked out,

transferring the forth editing plasmid into the strain ZM4-Cas12aΔ32Δ33Δ36 to obtain a strain named ZM4-Cas12aΔ32Δ33Δ36ΔTA::Cm that the toxin-antitoxin system operon on pZM39 has been replaced by a resistance gene (chloramphenicol, Cm),

transferring the third editing plasmid into the strain ZM4-Cas12aΔ32Δ33Δ36ΔTA::Cm to obtain a genetically modified ZM4-Cas12a with four endogenous plasmids, pZM32, pZM36, pZM33, and pZM39 knocked out, and

transferring the fifth editing plasmid into the genetically modified ZM4-Cas12a to obtain the genetically engineered strain of Z. mobilis.

Among embodiments, the first editing plasmid may carry a first CRISPR expression unit. The first CRISPR expression unit contains a leader region as SEQ ID NO.1, a repeat region as SEQ ID NO.2 and a first guide RNA as SEQ ID NO.3.

Among embodiments, the second editing plasmid may carry a second CRISPR expression unit. The second CRISPR expression unit contains a leader region as SEQ ID NO.1, a repeat region as SEQ ID NO.2 and a second guide RNA as SEQ ID NO.4.

Among embodiments, the third editing plasmid may carry a third CRISPR expression unit. The third CRISPR expression unit contains a leader region as SEQ ID NO.6, a repeat region as SEQ ID NO.7 and a third guide RNA as SEQ ID NO.5.

Among embodiments, the forth editing plasmid may carry a homology arm that is homologous to pZM39 and a resistance gene used to replace the toxin-antitoxin system gene operon on pZM39. In some examples, the resistance gene may be selected from ampicillin resistance gene, tetracycline resistance gene, chloramphenicol resistance gene, streptomycin resistance gene, kanamycin resistance gene, kanamycin resistance gene, mucin resistance gene, mycophenolic acid resistance gene, puromycin resistance gene, bleomycin resistance gene or neomycin resistance gene.

Among embodiments, the fifth editing plasmid may carry a fifth CRISPR expression unit. The fifth CRISPR expression unit contains a leader region as SEQ ID NO.6, a repeat region as SEQ ID NO.7 and a fifth guide RNA as SEQ ID NO.8.

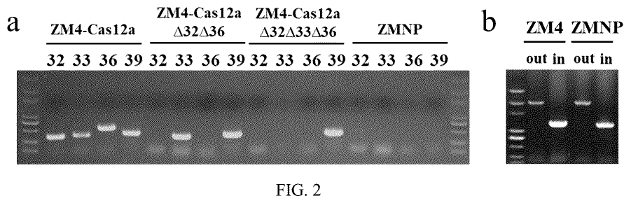

In some embodiments, ZMNP strains have been subjected to a series of phenotypic verification: morphological verification, transformation efficiency test, growth and glucose-ethanol metabolism test at 30° C. and 40° C., growth and ROS test under inhibitors, such as acetic acid, furfural and ethanol, and the utilization of secondary mother liquor medium, etc. The PCR verification results of stains in the construction process of ZMNP in accordance with embodiments are shown in FIG. 2.

(1) Morphological Verification of ZM4 and ZMNP

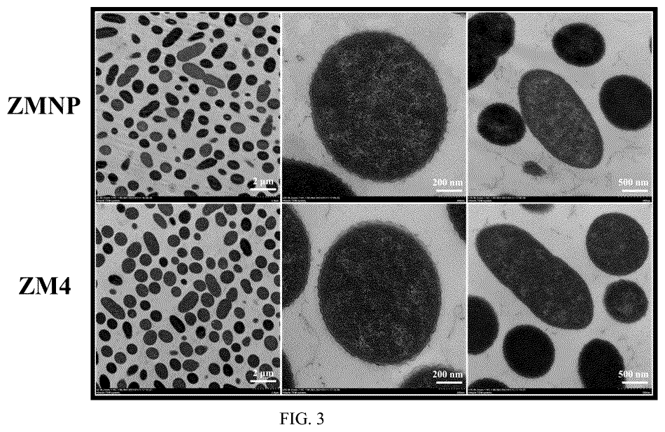

ZM4 and ZMNP can be morphologically visualized by transmission electron microscopy (TEM). As shown in FIG. 3, both ZM4 and ZMNP show short rod morphology, similar cellular morphology on transverse and longitudinal sections, and their cell sizes are about 2˜3 μm×1˜1.5 μm. This suggests that the knockouts of the four endogenous plasmids does not affect the cell shape of ZMNP.

(2) Test of Transformation Efficiency of ZMNP

As the ZMNP lacks the endogenous plasmid pZM32 which has a restriction-modification system that can recognize and cleave a sequence of 5′-GAAGNNNNNNNTCC, the transformation efficiency test of ZMNP should be determined in this disclosure. A plasmid named pEZ-HsdSp used for the test of transformation efficiency should be prepared.

50 ng of the pEZ-HsdSp plasmid in a demethylated state and a methylated state were electro-transferred into ZM4 and ZMNP, respectively, and the corresponding transformation efficiency was calculated by colony numbers.

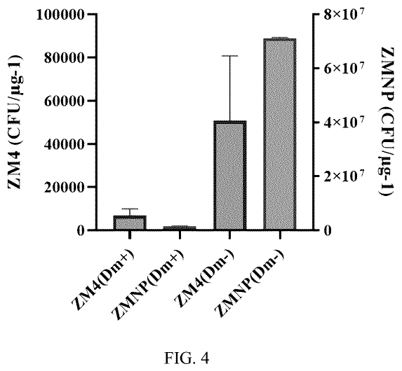

As shown in FIG. 4, both the transformation efficiencies of the methylated and demethylated plasmids of pEZ-HsdSp into the ZMNP show a 1,000-fold increase compared with ZM4.

(3) Growth and Glucose-Ethanol Metabolism Test of ZMNP in 30° C. and 40° C.

In this test, ZMNP and ZM4 were tested for growth and glucose-ethanol metabolism at 30° C. and 40° C., respectively. ZMNP and ZM4 were cultivated in 50 mL triangular flasks with a bottling quantity of 80% v/v of RMG5 at 30° C. and 40° C., respectively. At certain intervals during the fermentation, OD600 nm was tested, and 1 mL of the sample was stored at −80° C. After glucose had been consumed during the fermentation process, the pellets from the fermentation broth were collected to measure the changes of glucose and ethanol. Results of growth charts of ZMNP and ZM4 at 30° C. and 40° C. as well as the glucose consumption and ethanol generation at different times were analyzed.

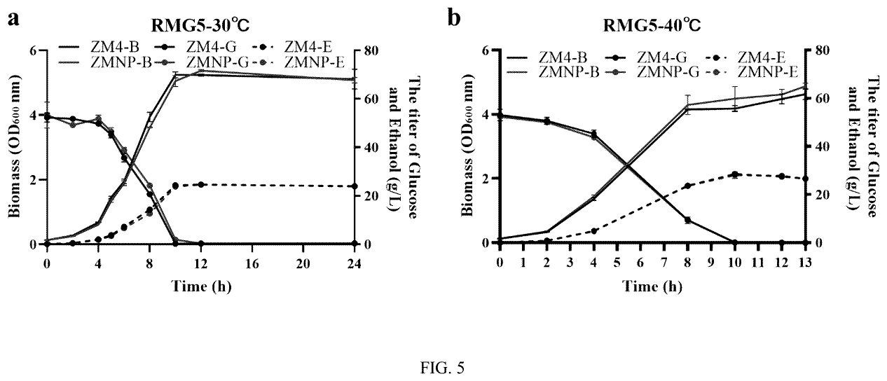

As shown in FIG. 5, the growth, glucose consumption, and ethanol production of ZMNP and ZM4 are similar at 30° C. and 40° C.

(4) Test of Growth and Intracellular ROS in the Media with Different Inhibitors

1) In Media without Containing Inhibitor

In this test, ZMNP and ZM4 were seeded into the medium named RMG5 and the basic medium of MMG5 at an initial inoculum of 0.1 OD600 nm, and cultivated in 50 mL triangular flasks with a bottling quantity of 80% v/v at a temperature of 30° C., and a stir speed of 100 rpm, respectively. Samples at different times were taken for OD600 nm test and growth curves were drawn. In RMG5, the 3 h sample was used for the ROS test. In MMG5, the 12 h sample was taken for the ROS test.

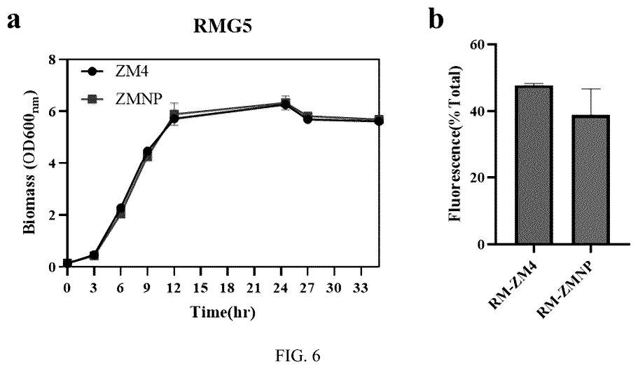

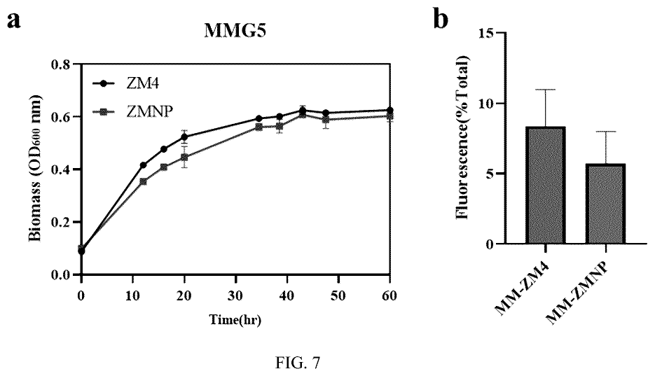

The results of the growth and ROS test in RMG5 are shown in FIG. 6. Results of growth and ROS test in MMG5 are shown in FIG. 7. These results suggest that the growth of ZMNP and ZM4 are similar in both media, and the intracellular ROS levels of ZMNP are lower than ZM4.

2) In Media Containing Acetic Acid Used as an Inhibitor

In this test, ZMNP and ZM4 were seeded into a medium named RMAce (RMG5 supplemented with 200 mM acetic acid) and a medium named MMAce (MMG5 supplemented with 200 mM acetic acid) at an initial inoculum of 0.1 OD600 nm, and cultivated in 50 mL triangular flasks with a bottling quantity of 80% v/v at a temperature of 30° C., and a stir speed of 100 rpm, respectively. Samples at different times were taken for OD600 nm test and the growth curves were drawn. In RMAce, a 3 h sample was used for the ROS test. In MMAce, a 12 h sample was taken for the ROS test.

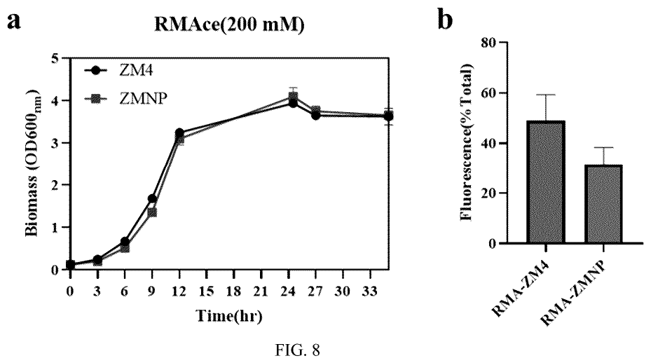

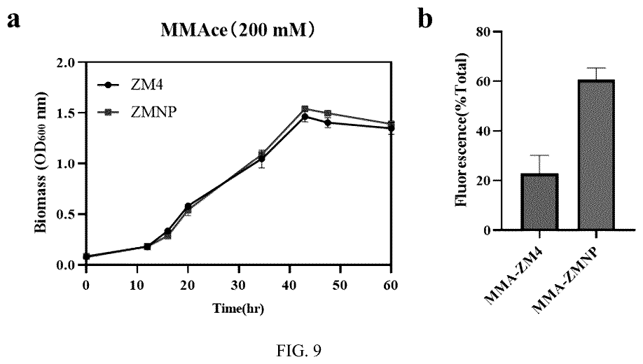

Results of growth and ROS test in RMAce are shown in FIG. 8. Results of the tests of growth and intracellular ROS in MMAce are shown in FIG. 9. These results suggest that the growth of ZMNP and ZM4 are similar in both mediums, and the intracellular ROS levels of ZMNP are lower than ZM4.

3) In Media Containing Furfural Used as an Inhibitor

In this test, ZMNP and ZM4 were seeded into a medium named RMF (RMG5 supplemented with 27 mM furfural) and a medium named MMF (MMG5 supplemented with 27 mM furfural) at an initial inoculum of 0.1 OD600 nm, and cultivated in 50 mL triangular flasks with a bottling quantity of 80% v/v at a temperature of 30° C., and a stir speed of 100 rpm, respectively. Samples at different times were taken for OD600 nm test and the growth curves were drawn. In RMF, a 3 h sample was used for the ROS test. In MMF, a 12 h sample was taken for the ROS test.

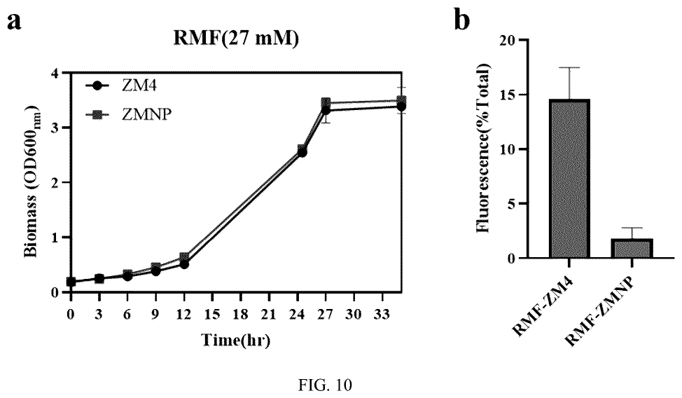

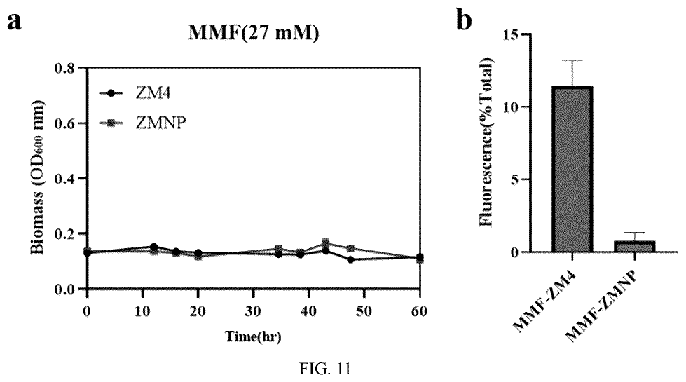

Results of growth and ROS test under RMF condition are shown in FIG. 10. Results of the measurement of growth and intracellular ROS under MMF conditions are shown in FIG. 11. These results suggest that the growth of ZMNP and ZM4 are similar in both media, and the intracellular ROS levels of ZMNP are lower than ZM4.

4) In Media Containing Ethanol Used as an Inhibitor

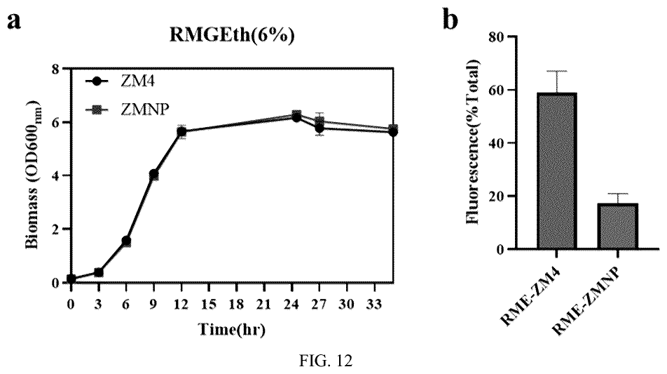

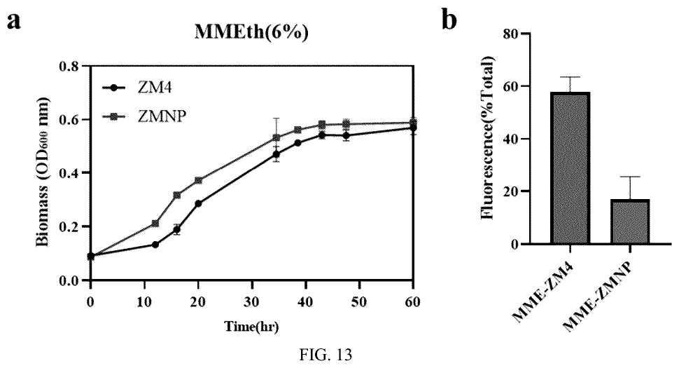

In this test, ZMNP and ZM4 were seeded into a medium named RMEth (RMG5 supplemented with 6% v/v ethanol) and a medium named MMEth (MMG5 supplemented with 6% v/v ethanol) at an initial inoculum of 0.1 OD600 nm, and cultivated in 50 mL triangular flasks with a bottling quantity of 80% v/v at a temperature of 30° C., and a stir speed of 100 rpm, respectively. Samples at different times were taken for OD600 nm test and the growth curves were drawn. In RMEth, the 3 h sample was used for the ROS test. In MMEth, the 12 h sample was taken for the ROS test. The results of the growth and ROS test in RMEth are shown in FIG. 12. The results of the growth and ROS test in MMEth are shown in FIG. 13. The growth of ZMNP and ZM4 is similar in both media and the intracellular ROS levels of ZMNP are lower than ZM4.

(5) Secondary Mother Liquor Utilization of ZMNP

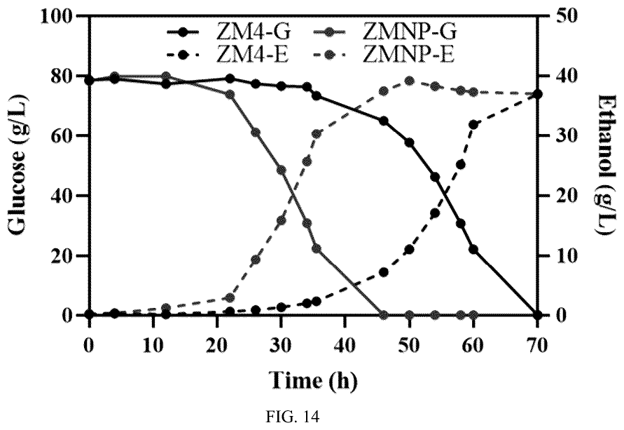

In this test, the xylose mother liquor was provided by Zhejiang Huakang Pharmaceutical Co., Ltd. (Kaihua, Zhejiang, China). The secondary mother liquor with a high sugar content was made by a hydrolysis of corn cob, and a sugar crystallization of the remaining residual liquor from the hydrolysis. The main components of the secondary mother liquor include xylose (103.98 g/L), glucose (196.18 g/L), arabinose (239.2 g/L), mannose (45.75 g/L), acetic acid (1.16 g/L), furfural (4.44 g/L), and HMF (0.87 g/L). A ⅓ of the secondary mother liquor medium was used in this test. This ⅓ secondary mother liquor medium was prepared as follows: 270 mL of xylose secondary mother liquor, 51 μL of 10×RM⁻ mother liquor (100 g/L yeast extract, 20 g/L of KH₂PO₄), and 340 mL of pure water were mixed and filtered for sterilization. ZMNP and ZM4 were seeded into a 1 L fermenter containing 500 mL of ⅓ secondary mother liquor medium at an initial inoculum of 0.1 OD600 nm, respectively, and cultivated at a temperature of 30° C. and a stir speed of 100 rpm, and controlled pH for 4.9 with 2 M potassium hydroxide. Samples at different times were used to test the glucose and ethanol content. The results of ZMNP and ZM4 glucose consumption and ethanol production are shown in FIG. 14.

The preparation process of the first editing plasmid, second editing plasmid, third editing plasmid, fourth editing plasmid, and fifth editing plasmid used in the above embodiments, as well as the editing process of ZM4-Cas12a gene-edited by these plasmids to obtain ZMNP, will be described in detail embodiments below.

1. Preparations of the First Editing Plasmid and the Second Editing Plasmid

Four guide RNAs targeting the replicase genes ZMOp32×017, ZMOp33×028, ZMOp36×036, ZMOp39×032 of these four ZM4 endogenous plasmids, shown in a order as in SEQ ID NO.3, SEQ ID NO.4, SEQ ID NO.5 and SEQ ID NO.8, were synthesized, respectively. These four guide RNAs could break the DNA double strands of endogenous plasmid. Because of the lack of a non-homologous end-joining repair system in Z. mobilis, the broken DNA could not be continuously inherited by the next generation for the knockout of endogenous plasmids. So, the first editing plasmid was used to targetedly knock out the pZM32 and the pZM36, the second editing plasmid was used to targetedly knock out the pZM33. The third editing plasmid was used to targetedly knock out the pZM39.

Methods of plasmid preparation, gene edit and transformation to ZM4-Cas12a of the first and the second editing plasmid were implemented according to CN110358767A.

2. Preparation of the Third Editing Plasmid

In examples, the preparation of the third editing plasmid was provided as follows:
(1) A 32 bp downstream sequence of the PAM CCC site of a replicase gene of ZMOp39×032 was set as targeted primers for amplifying a guide RNA. The guide RNA was set to guide the cleavage of the target site by a nuclease. Primers of the guide RNA 39-032-gR-F: 5′-gaaagtaaccagtccttttatcgacaggctaggccg-3′, SEQ ID NO.9, and 39-032-gR-R: 5′ gaaccggcctagcctgtcgataaaaggactggttac-3′, SEQ ID NO.10, were synthesized. The first four oligonucleotides of each primer were set as the adaptor and paired with a digested vector.
(2) The primers of the guide RNA were ligated with a kanamycin-editing plasmid vector containing a Type I-F CRISPR-Cas expression unit (CN110408642A). And the process included:
1) linearizing a vector by using the restriction enzyme Bsa I;
2) annealing the primers in a case of 10 μL reaction system contain 10 μM of each primer with 1 μL, and remaining water;
3) denaturing the guide primers at a temperature of 95° C. for 5 min, and cooling to room temperature;
4) ligating the annealed products with the linearized vector by using T4 DNA ligase;
5) transferring the ligation product into E. coli DH5α by chemical transformation for plasmid construction;
6) screening and verifying the transformants by a colony PCR and a sequencing analysis. The reaction systems of Bsa I digestion and T4 DNA ligation are shown in Table 1 and Table 2.

TABLE 1
Reaction system of Bsa I digestion

| Reagent | Volume | |
|---|---|---|
| a kanamycin-editing plasmid vector | 2 | μg |
| Bsa I | 0.5 | μL |
| Cutsmart | 5 | μL |

| | |
|---|---|
| ddH₂O | Up to 50 μL |

37° C., 3 h

TABLE 2
Reaction system of T4 DNA ligation

| Reagent | Volume | |
|---|---|---|
| Annealed oligonucleotide | 2 | μL |
| Vector | 2 | μg |
| T4 ligase | 0.5 | μL |
| T4 ligase buffer | 5 | μL |

| | |
|---|---|
| ddH₂O | Up to 50 μL |

22° C., 3 h

3. Preparation of the Fourth Editing Plasmid

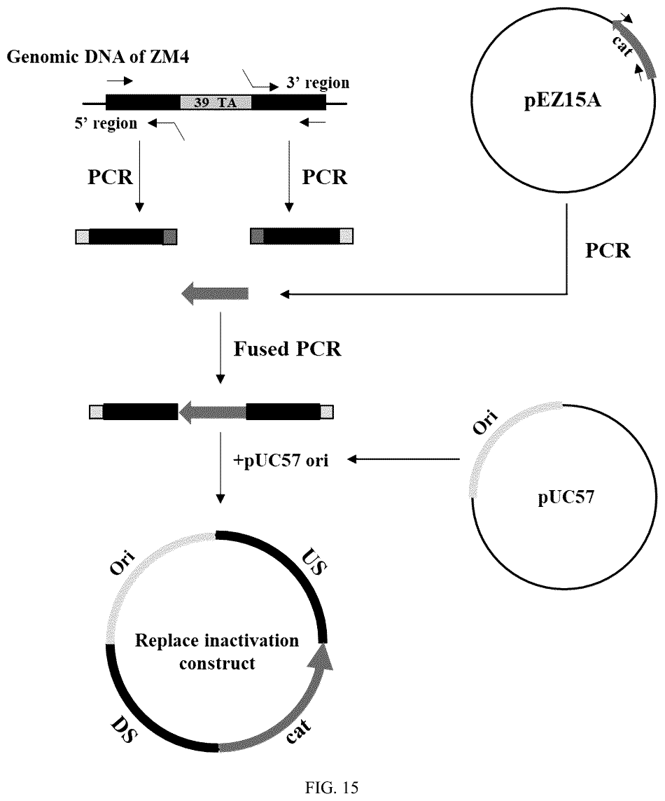

In some embodiments, the fourth editing plasmid was used for replacing the T-A system genes on pZM39. As shown in FIG. 15, the fourth editing plasmid was prepared by taking the steps including (1) to (6).
(1) Preparation of the ZM4 genome: collecting 2 mL of overnight cultured ZM4 broth and extracting by using a bacterial genome extraction kit.
(2) Preparation of homology arms US and DS: amplifying by a PCR using ZM4 genome as template, using primers shown in SEQ ID NO.11˜12, and gel extracting a 1 kb sequence upstream of ZMOp39×020 (named US); amplifying by a PCR using ZM4 genome as template, using primers shown in SEQ ID NO.13˜14, and gel extracting a 1 kb downstream of ZMOp39×023 (named DS).
(3) Preparation of a chloramphenicol gene: amplifying by PCR using primers shown in SEQ ID NO.15˜16 from a plasmid named pEZ15A, and gel extracting a fragment of chloramphenicol gene.
(4) Preparation of a fragment of Ori: amplifying by PCR using primers shown in SEQ ID NO.17˜18 from a plasmid named pUC57, and gel extracting a fragment of Ori.
(5) Preparation of a ligation product: ligating the US, the fragment of chloramphenicol gene and the DS by a Overlap PCR shown in Table 3 and Table 4, and gel extracting to get a first long fragment.

TABLE 3
Reaction system of Overlap PCR

| Reagent | Volume | |
|---|---|---|
| US-F-primer (10 μM) | 2.4 | μL |
| DS-R-primer (10 μM) | 2.4 | μL |
| 2× PrimerSTAR (Takara) | 30 | μL |
| US fragment | 80 | ng |
| DS fragment | 80 | ng |
| Cat fragment | 80 | ng |
| ddH₂O | Up to 60 μL | |
| Total volume | 60 | μL |

TABLE 4
Reaction procedure of Overlap PCR

| Temperature | Time | Cycles |
|---|---|---|
| 98° C. | 3 min | |
| 98° C. | 10 s | 10 |
| 50° C. | 20 s | |
| 72° C. | 20 s | |
| 72° C. | 1 min | |

Add primers
98° C. 3 min

| | | |
|---|---|---|
| 98° C. | 10 s | 25 |
| 55° C. | 10 s | |
| 72° C. | 30 s | |
| 16° C. | hold | |

Primers are added at the last 25 cycles

(6) Preparation of the fourth editing plasmid: digesting the first long fragment and the fragment of Ori in (4) and (5) with T5 exonuclease (shown in Table 5), transforming into a competent cell of E. coli DH5α, verifying positive clones on the plates by a PCR, extracting the fourth editing plasmids after overnight culture (Plasmid extraction was performed as the plasmid extraction kit).

TABLE 5
Gibson assembly reaction system by using T5 exonuclease

| Reagent | Volume | |
|---|---|---|
| DNA fragment | 0.12 | pM |
| Vector | 0.04 | pM |
| 10× Buffer 4 (Thermo) | 0.5 | μL |
| T5 Exonuclease | 0.5 | U |

| | |
|---|---|
| ddH₂O | To 5 μL |

4. Preparation of the Fifth Editing Plasmid

(1) Select of a Target Site

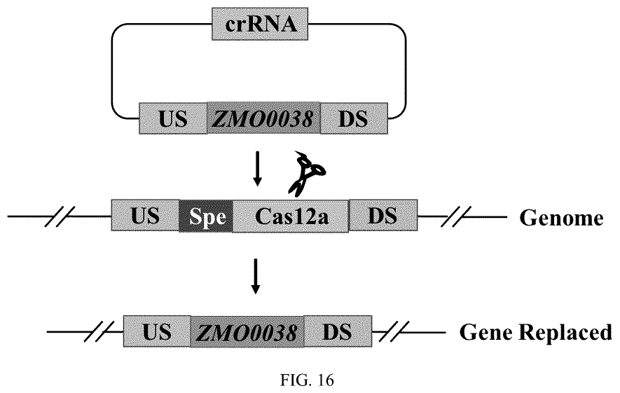

As shown in FIG. 16, in this step, the target site was selected from the Cas12a gene in the genome of ZM4-Cas12a, and the spectinomycin gene from the upstream of the target site was replaced by ZMO0038. Primers of a guide RNA for cutting the target site by a nuclease were selected from the 32 bp downstream sequence of the PAM site “CCC” of the Cas12a gene. Primers of the guide RNA, Cas12a-gR-F: 5′-gaaatgcgttttgaactgattccgcagggtaaaacc-3′, SEQ ID NO.19, Cas12a-gR-R: 5′-gaacggttttaccctgcggaatcagttcaaaacgca-3′, SEQ ID NO.20, were synthesized. The first four oligonucleotides of each primer was set as the adaptor and paired with the digested vector.

(2) Preparation of a Targeted Plasmid

In this step, the primers of the guide RNA were ligated into a vector containing a Type I-F CRISPR-Cas expression unit (CN110408642A) for editing chloramphenicol. The processes included: (1) linearizing the vector by using the restriction enzyme Bsa I, (2) annealing the primers of the guide RNA in a case of 10 μL reaction system containing 10 μM of each primer with 1 μL, and remaining water, and denaturing the primers of the guide RNA at 95° C. for 5 min, and cooling to room temperature, (4) ligating the annealed products with the linearized vector using a T4 DNA ligase, (5) transferring the ligation product into E. coli DH5α by chemical transformation, (6) screening and verifying the transformants by a colony PCR and a sequencing analysis.

(3) Preparation of the Fifth Editing Plasmid

In this step, a fragment of 1-kb upstream of Cas12a was amplified by a PCR using Cas12a-US-F (as in SEQ ID NO.21) and Cas12a-US-R (as in SEQ ID NO.22) as primers. A fragment of 1-kb downstream of Cas12a was amplified by a PCR using Cas12a-DS-F (as in SEQ ID NO.23) and Cas12a-DS-R (as in SEQ ID NO.24) as primers. A sequence of ZMO0038 was amplified by a PCR using 0038-F (as in SEQ ID NO.25) and 0038-R (as in SEQ ID NO.26) as primers. The fragment of 1 kb upstream of Cas12a, the sequence of ZMO0038, and the fragment of 1 kb downstream of Cas12a were connected into a second-long fragment by an Overlap PCR. The reaction system of the Overlap PCR is shown in Table 3, and the reaction procedure of Overlap PCR is shown in Table 4.

The targeted plasmid prepared in the previous step was amplified by a reverse PCR by using primers shown in SEQ ID NO. 2728, to obtain a fragment of the targeted plasmid. The procedure of the reverse PCR was set as: 98° C. pre-denaturation for 3 min, 98° C. denaturation for 10 s, 55° C. annealing for 10 s, 72° C. extension for a total of 30 cycles (set according to 10 s/kb by fragment length), 72° C. for 5 min after the last cycle. The second long fragment in the previous step and the fragment of the targeted plasmid was further ligated by a Gibson assembly and transferred to the E. coli DH5α. The transformants were screened by a colony PCR and verified by a sequencing analysis to obtain the fifth editing plasmid.

5. Transformation of the Gene Editing Plasmids

In some embodiments, methods for transferring the first, second, third, fourth, and fifth editing plasmid may be essentially the same. The process of the transformation was roughly as follows:

(1) Preparation of Competent Cells

100 μL strains which stored in a −80° C. refrigerator were inoculated into 1 mL RMG5, and then incubated at 30° C. After incubation to turbidity, the culture was transferred to a blue cap bottle of 250 mL containing a liquid medium RMG5 of 200 mL to allow the initial OD600 nm from 0.025 to 0.3, and incubated at 30° C. When OD600 nm exceeded 0.3, the competent cells in the culture were collected at 100 rpm at room temperature, then washed once with sterile water and twice with 10% glycerin. Finally, the competent cells were slowly resuspended with 1˜2 mL of 10% glycerol and divided into 55 μL into 1.5 mL EP tubes.

(2) Electro-Transformation Process

200 ng of the first editing plasmid, 200 ng of the second editing plasmid, 200 ng of the third editing plasmid, 200 ng of the fourth editing plasmid, and 500 ng of the fifth editing plasmid were respectively added to 1.5 mL EP tubes containing 55 μL competent cells, gently mixed well and transferred to 1 mm gap electroporation cuvettes. The electro-transformation procedure was: 200Ω, with capacitance of 25 μF, and voltage of 1.6 KV. Place the electro-poration cuvettes into an electro-poration instrument for electro-transformation. Immediately after electro-transformation, 1 mL RMG5 of the liquid medium was added, mixed and transferred to sterile EP tubes, sealed with sealer films, and incubated at 30° C. for 4˜6 h. 100 μL of the bacterial solution was taken and spread evenly to RMG5 supplemented with a Cm plate (with an RMG5 supplemented with 100 μg/mL chloramphenicol), whereas, an RMG5 supplemented with Km plate (with a supplement of 300 μg/mL kanamycin into RMG5) was applied for spreading the bacterial solution that has been transferred with the third editing plasmid. The plates were sealed with sealer films and reversely placed in an incubator at 30° C.

(3) Colony PCR Verification Process

In this step, after growing on the plate, the colonies were verified by a colony PCR to verify the transferred gene-editing plasmids. The reaction system of the colony PCR is shown in Table 6, and the reaction procedure of colony PCR is shown in Table 7.

TABLE 6
Reaction system of colony PCR

| Reagent | Volume | |
|---|---|---|
| F-primer (10 μM) | 0.4 | μL |
| R-primer (10 μM) | 0.4 | μL |
| 2× T5 Super PCR Mix (Tsingke) | 5 | μL |
| Template (single colony dissolved in 10 μL ddH₂O) | 1 | μL |
| ddH₂O | 3.2 | μL |
| Total volume | 10 | μL |

TABLE 7
Reaction procedure of colony PCR

| | | |
|---|---|---|
| Temperature | Time | Cycles |

| | | | |
|---|---|---|---|
| 98° C. | 3 | min | |
| 98° C. | 10 | s | 29 |
| 55° C. | 10 | s | |
| 72° C. | 10 | s | |
| 72° C. | 3 | min | |

| | |
|---|---|
| 16° C. | hold |

(4) Knockout of the Gene Editing Plasmids

Strains with successful knockout of endogenous plasmids were seeded in a liquid medium named RMG5 without containing antibiotics. After growing to turbidity, 100 μL of bacterial solution was transferred to 1 mL of fresh RMG5 liquid medium for 4-5 generations, and 100 μL of bacterial solution was diluted and plated on RMG5. After growing on the plate, colonies were verified by PCR to verify the gene-editing plasmids. If the electrophoresis of the PCR does not have PCR products, the editing plasmid may be lost. Colonies that did not have PCR products were inoculated into the liquid medium of RMG5 and RMG5 supplemented with Cm, respectively, and placed for standing cultivating at 30° C. The next day, cultures under the two media were observed. If the culture became cloudy in the liquid medium of RMG5, but can not clear in the liquid medium of RMG5 supplemented with Cm, it could confirm that the gene editing plasmid has been knocked out, which was then further verified by colony PCR. Among them, the knockout of the third editing plasmid was verified under the liquid medium of RMG5 supplemented with Kan.

6. Examples of Knockouts of the Four Endogenous Plasmids

In examples, the first editing plasmid targeting ZMOp32×017 was electro-transferred into ZM4-Cas12a and plated on the plate of RMG5 supplemented with Cm. The resulting transformants were validated by a colony PCR using 32-check-F/R (SEQ ID NO. 31˜32), 33-check-F/R (SEQ ID NO.33˜34), 36-check-F/R (SEQ ID NO.35˜36), 39-check-F/R (SEQ ID NO.37˜38) as primers, respectively. And the PCR results of ZM4-Cas12a were used as control. The presence of endogenous plasmids was observed according to the PCR results. The Colony PCRs showed that the knockout of pZM32 was accompanied by the knockout of pZM36. Colonies were serially passaged in a liquid medium of RMG5 to obtain a strain named ZM4-Cas12aΔ32Δ36 without containing the gene editing plasmids. The competent cells of ZM4-Cas12aΔ32Δ36 were prepared for subsequent use.

The second editing plasmid targeting ZMOp33×028 was electro-transferred into ZM4-Cas12aΔ32Δ36 and plated on the plate of RMG5 supplemented with Cm. The resulting transformants were validated by a colony PCR using 33-check-F/R, 32-check-F/R, 36-check-F/R, 39-check-F/R as primers, respectively. And the PCR results of ZM4-Cas12a were used as control. The presence of endogenous plasmids was observed according to the PCR results. Colony PCR showed the knockout of pZM33 on the previous basis. Colonies were serially passaged in a liquid medium of RMG5 to obtain a strain named ZM4-Cas12aΔ32Δ33Δ36 without containing editing plasmids. The competent cells of ZM4-Cas12aΔ32Δ33Δ36 were prepared for subsequent use.

The fourth gene editing plasmid used to replace the toxin-antitoxin system sense on pZM39 was electro-transferred into ZM4-Cas12aΔ32Δ33Δ36. The resulting transformants were validated by a colony PCR using 39-TA-check-F/R and confirmed that the T-A genes were knocked out, and cultivated in a liquid medium of RMG5. And the competent cells were prepared.

The third editing plasmid targeting ZMOpp39×032 was electro-transferred into the competent cells of the previous step and plated on plate RMG5 supplemented with Kan. The resulting transformants were validated by colony PCR using 39-check-F/R, 32-check-F/R, 33-check-F/R as primers, respectively. And the PCR results of ZM4-Cas12a were used as control. The presence of endogenous plasmids was observed according to the PCR results. Colony PCR showed the knockout of pZM33 on the previous basis. Colonies were serially passaged in a liquid medium of RMG5 to obtain a strain ZMNP-Cas12a without containing the first, second, third, and fourth editing plasmids. The competent cells of this strain were prepared.

The fifth editing plasmid was used to targetedly knock out Cas12a and the spectinomycin gene was electro-transferred into the competent cells of the previous step and plated on plate RMG5 supplemented with Cm. The resulting transformants were validated by colony PCR using 0038-out-F/R (SEQ ID NO.39, 40), and 0038-out-F/in-R2 (SEQ ID NO.39, 41) as primers, respectively. Colonies that their Cas12a and the spectinomycin gene were successfully replaced by ZMO0038, and were serially passaged in RMG5 liquid medium to obtain a strain named ZMNP without containing the first, second, third, fourth, and fifth editing plasmids.

7. Test of Transformation Efficiency of ZMNP

(1) Preparation of a Plasmid Named pEZ-HsdSp for Test of Transformation Efficiency

The sequence of 5′ GAAGNNNNNNNTCC was placed at the 5′ end of the forward primer of HsdSp-F (SEQ ID NO.42) and the reverse primer of HsdSp-R (SEQ ID NO.43), respectively. A sequence of pEZ15A was reverse amplified by a PCR with these primers, and subjected to electrophoresis and gel purification. The plasmid of pEZ-HsdSp was generated by a Gibson assembly based on T5 exonuclease for ligation of the sequence of pEZ15A, and a sequencing verification.

(2) Test of Transformation Efficiency

Methylated and demethylated pEZ-HsdSp plasmids were extracted from E. coli DH5α and Trans110 (its methyltransferase has been knocked out, Trans110 purchased from Tsingke Biotechnology Co., Ltd.), respectively.

The competent cells of ZM4 (Z. mobilis subsp. ZM4 (wild-type) ATCC3182, purchased from ATCC) and ZMNP were placed and thawed on ice. 200 ng of the pEZ-HsdSp was respectively added to ZM4 and ZMNP, gently mixed well, and transferred to two electroporation cuvettes. Then, the two electroporation cuvettes were placed into an electroporation instrument for electro-transformation. Immediately after electro-transformation, the electro-cultures were resuspended in a liquid medium of RMG5, placed for cultivation at 30° C. for 4 to 6 h, and then plated with 100 μL to the RMG5 supplemented with spectinomycin at different dilutions. After incubation, the number of colonies (CFU) on the plate was counted.

The transformation efficiency is calculated as:

CFU/μg-1 DNA=(Cp/Tp)×(Vt/Vp),

Where “Cp” is the colony number counted on selective plates; “Tp” is the total amount of plasmid DNA (μg) used here; “Vt” is the total transformation volume (μL); “Vp” is the volume (μL) plated.

8. Fermentation Test

The resulting target strain ZMNP and the wild-type strain ZM4 were tested for fermentation in RMG5.

First of all, ZMNP and ZM4 were transferred into a frozen stock tube containing 1 mL RMG5, then activated to turbidity in an incubator at 30° C., and then transferred into a 100 mL triangular bottle with 80 mL liquid medium of RMG5 as a fermentation seed to incubate to mid-late log at 30° C.

Further, the fermentation seed was switched to a 50 mL triangular flask filled with a 40 mL liquid medium of RMG5, and the initial OD600 nm value of the fermentation broth was controlled at 0.1.

During the fermentation process, the optical density of OD600 nm was measured by a UV spectrophotometer. And the cell growth measured at different times was determined. And The fermentation broth samples obtained at different times were collected and tested by HPLC for the content of glucose and ethanol.

The test conditions for the HPLC include Liquid Chromatography: Agilent 1100 (LC-20AD), Differential Refractive Index Detector (RID-10A), Organic acid HPLC column (Bio-Rad Aminex HPX-87H, 300 mm×7.8 mm), pool temperature at 40° C., column temperature of 60° C., the mobile phase is set to 5 mM of sulfuric acid, the elution flow rate is set to 0.5 mL/min, the initial flow rate of the instrument is set to 0.2 mL/min, the flow rate is gradually increased at 0.1 mL/min to 0.5 mL/min after the column pressure is stabilized, the sample injection volume is set to 20 μL. After the test, export the data and make the figure.

Among these, the configuration steps of the mobile phase include: taking 1.41 mL of concentrated sulfuric acid to 5 L, mixing well with ultrapure water to 5 L, filtering with 0.45 μm aqueous filter membrane, dividing the filtered mobile phase into 1 L blue cap bottles for ultrasonic degassing for 20˜30 min, and after returning to room temperature for use.

9. Test of Intracellular ROS

In this test, the intracellular ROS is determined by using a ROS Assay Kit (Beyotime Biotechnology, China). And a fluorescent probe of DCFH-DA is used for the test. The DCFH-DA does not produce fluorescence by itself and can freely cross the cell membrane and. After entering the cell, the DCFH-DA can be hydrolyzed by intra-cellular esterases to generate DCFH. And The DVFH cannot pass through the cell membrane, so that the probe can be easily packed into the cell. The intracellular ROS can oxidize the non-fluorescent DCFH to generate a fluorescent DCF. The detection of the fluorescent DCF reveals the intra-cellular level of reactive oxygen species. Among the test, Rosup is served as a positive control reagent.

During the growth curve determination, 0.6 OD600 nm of cells were collected from cultures grown for 3 h in RMG5 and for 12 h in MMG5 and washed once with PBS, and then resuspended with 500 μL PBS, added with 1 μL DCFH-DA, mixed and incubated at 30° C., 100 rpm for 1 h. And then, the solution from the incubation was centrifuged, and the supernatant from the centrifugation was removed and washed three times with PBS and resuspended in 300 μL PBS. As comparisons, the DCFH-DA was not added during the treatment of the negative control sample, and another 6 μL Rosup should be added during the treatment of the positive control sample. After these steps, the samples were subsequently examined by a flow cytometry (CytoFLEX FCM, Beckman coulter, CA, USA), with a wavelength of 488 nm for exciting light and a wavelength of 525 nm for emitted light. Cells with fluorescence intensity ranging from 10³ to 10⁵ were selected, and 20,000 cells per sample were analyzed.

10. Fermentation Test of Secondary Mother Liquor

The glycerobacteriums of ZM4 and ZMNP were inoculated into two frozen stock tubes containing 1 mL RMG5, respectively, then activated to turbidity in the 30° C. incubator, transferred into a 100 mL triangular bottle with 80 mL medium named RMG5 as a fermentation seed to incubate to mid-late log at 30° C. It was further inoculated to a 1 L fermentor containing 500 mL ⅓ secondary mother liquor medium, and the initial OD600 nm was controlled at 0.1. A 2 M potassium hydroxide catheter was inserted into the fermentor to control the pH at 4.9. The fermentation conditions included controlling the pH at 4.9 by inserting a 2 M potassium hydroxide catheter into the fermentor, setting the temperature to 30° C. and stirring the culture with 100 rpm. During the fermentation, samples obtained at different time were collected, and then the content of glucose and ethanol were tested by HPLC with the same test process as above.

The above is only the preferred embodiments of this disclosure and is not intended to limit this disclosure. Any modification, equivalent replacement, improvement, etc., made within the spirit and principle of this disclosure shall be included in the scope of this disclosure.

1. A genetically engineered strain of Z. mobilis, named ZMNP, is obtained by knockouts of four endogenous plasmids, pZM32, pZM36, pZM33, pZM39, a nuclease Cas12a gene and a spectinomycin gene from a strain named ZM4-Cas12a;
wherein, the stain ZM4-Cas12a is a recombinant strain prepared by integrating the nuclease Cas12a gene, which is derived from F. novicida, and the spectinomycin gene into a ZMO0038 site on a genome of strain ZM4 by homologous recombination, and using an inducible promoter Ptet to control a expression of the nuclease Cas12a gene, a number in Genbank of pZM32 is No. CP023678, a number in Genbank of pZM33 is No. NZ_P023679, a number in Genbank of pZM36 is No. CP023680 and a number in Genbank of pZM39 is No. CP023681.

2. A method for preparing a genetically engineered strain of Z. mobilis, wherein the genetically engineered strain of Z. mobilis named ZMNP, is obtained by knockouts of four endogenous plasmids, pZM32, pZM36, pZM33, pZM39, a nuclease Cas12a gene and a spectinomycin gene; the stain ZM4-Cas12a is a recombinant strain prepared by integrating the nuclease Cas12a gene, which is derived from F. novicida, and the spectinomycin gene into a ZMO0038 site on a genome of strain ZM4 by homologous recombination, and using an inducible promoter Ptet to control a expression of the nuclease Cas12a gene, a number in Genbank of pZM32 is No. CP023678, a number in Genbank of pZM33 is No. NZ_P023679, a number in Genbank of pZM36 is No. CP023680 and a number in Genbank of pZM39 is No. CP023681,
the method comprises:
preparing a first editing plasmid used to targetedly knock out the pZM32 and the pZM36, a second editing plasmid used to targetedly knock out the pZM33, a third editing plasmid used to targetedly knock out the pZM39, a fourth editing plasmid used to replace a toxin-antitoxin operon of the pZM39, and a fifth editing plasmid used to targetedly knock out the Cas12a gene and the spectinomycin gene;
transferring the first editing plasmid into the ZM4-Cas12a to obtain a strain named ZM4-Cas12aΔ32Δ36, that the endogenous plasmids of the pZM32 and the pZM36 have been targetedly knock out;
transferring the second editing plasmid into the ZM4-Cas12aΔ32Δ36 to obtain a strain named ZM4-Cas12aΔ32Δ33Δ36, that the endogenous plasmids of the pZM32, the pZM36 and the pZM33 have been targetedly knocked out;
transferring the fourth editing plasmid into the ZM4-Cas12aΔ32Δ33Δ36 to obtain a strain named ZM4-Cas12aΔ32Δ33Δ36ΔTA::Cm, that the toxin-antitoxin operon of the pZM39 has been replaced by a chloramphenicol gene;
transferring the third editing plasmid into the ZM4-Cas12aΔ32Δ33Δ36ΔTA::Cm to obtain a stain of ZM4-Cas12a, that the four endogenous plasmids, the pZM32, the pZM36, the pZM33, and the pZM39 have been knocked out; and
transferring the fifth editing plasmid into the ZM4-Cas12a to obtain the ZMNP, that the four endogenous plasmids, pZM32, pZM36, pZM33, and pZM39 have been knocked out;
wherein, the first editing plasmid and the second editing plasmid are prepared by using a CRISPR-Cas12a gene edit system, the third editing plasmid is prepared by using an endogenous type I-F CRISPR-Cas gene editing system of Zymomonas mobilis,
wherein, a method of preparing the fourth editing plasmid comprises:
amplifying by using primers shown in SEQ ID NO.11˜12 and using the genome of strain ZM4 as a template to get an upstream 1 kb fragment of ZMOp39×020 named US, and using primers shown in SEQ ID NO.13˜14 and using the genome of strain ZM4 as a template to get a downstream 1 kb fragment of ZMOp39=023 named DS;
amplifying by using primers shown in SEQ ID NO.15˜16 from a plasmid named pEZ15A, and gel extracting a fragment of chloramphenicol gene;
amplifying by using primers shown in SEQ ID NO.17˜18 from a plasmid named pUC57, and gel extracting a fragment of Ori;
connecting the US, the fragment of chloramphenicol gene and the DS by an Overlap PCR, and gel extracting to get a first long fragment; and
digesting the first long fragment and the fragment of Ori with T5 enzyme, and transferring into a competent cell of E. coli DH5α, screening positive clones by a PCR, and extracting the fourth editing plasmid after overnight culture;
wherein, a method of preparing the fifth editing plasmid comprises:
ligating annealed primers for guiding a Cas12a gene shown in SEQ ID NO.19˜20 into a chloramphenicol edit vector contained a Type I-F CRISPR-Cas expression unit to get a targeted plasmid;
amplifying a fragment of 1-kb upstream of the Cas12a by using a primer named Cas12a-US-F as shown in SEQ ID NO.21 and a primer named Cas12a-US-R as shown in SEQ ID NO.22;
amplifying a fragment of 1-kb upstream of the Cas12a by using a primer named Cas12a-DS-F as shown in SEQ ID NO.23 and a primer named Cas12a-DS-R as shown in SEQ ID NO.24;
amplifying a fragment of ZMO0038 by using a primer named 0038-F as shown in SEQ ID NO.25 and a primer named 0038-R as shown in SEQ ID NO.26;
connecting the fragment of 1-kb upstream of Cas12a, the fragment of ZMO0038 and the fragment of 1-kb downstream of Cas12a, and gel extracting to get a second long fragment;
reversely amplifying the targeted plasmid by using primers as shown in SEQ ID NO.27˜28 to get a fragment of the targeted plasmid; and
ligating the second long fragment and the fragment of the targeted plasmid by a Gibson assembly, transferring into E. coli DH5α, screening positive clones by a PCR, and verifying by sequencing analysis to obtain the fifth editing plasmid.

3. The method according to claim 2, wherein, the first editing plasmid carries a first CRISPR expression unit, the first CRISPR expression unit has a leader region as SEQ ID NO.1, a repeat region as described in SEQ ID NO.2, and a first guide RNA as SEQ ID NO.3;
the second editing plasmid carries a second CRISPR expression unit, the second CRISPR expression unit has a leader region as SEQ ID NO.1, a repeat region as described in SEQ ID NO.2, and a second guide RNA as SEQ ID NO.4;
the third editing plasmid carries a third CRISPR expression unit, the third CRISPR expression unit has a leader region as SEQ ID NO.6, a repeat region as described in SEQ ID NO.7, and a third guide RNA as SEQ ID NO.5;
the fourth editing plasmid carries a homology arm with the pZM39 and a resistance gene used to replace the toxin-antitoxin system operon on pZM39;
the fifth editing plasmid carries a fifth CRISPR expression unit, the fifth CRISPR expression unit has a leader region as SEQ ID NO.6, a repeat region as described in SEQ ID NO.7, and a fifth guide RNA as SEQ ID NO.8.

4. The method according to claim 2, wherein, the resistance gene is selected from ampicillin resistance gene, tetracycline resistance gene, chloramphenicol resistance gene, streptomycin resistance gene, monomycin resistance gene, kanamycin resistance gene, mycophenolate resistance gene, puromycin resistance gene, bleomycin resistance gene or neomycin resistance gene.

5. A use of the genetically engineered strain described in claim 1 in the preparation of ethanol fermentation chassis bacteria.


SEQUENCE LISTING (41 sequences)

SEQ ID NO: 1 - DNA, 35 bp, unidentified
  source 1..35: mol_type = other DNA
ttgacagctagctcagtcctaggtataatgctagc

SEQ ID NO: 2 - DNA, 19 bp, unidentified
  source 1..19: mol_type = other DNA
aatttctactcttgtagat

SEQ ID NO: 3 - DNA, 23 bp, unidentified
  source 1..23: mol_type = other DNA
gagcgtgtgatgagccggaggaa

SEQ ID NO: 4 - DNA, 23 bp, unidentified
  source 1..23: mol_type = other DNA
cccccgatatttctttcatgaat

SEQ ID NO: 5 - DNA, 32 bp, unidentified
  source 1..32: mol_type = other DNA
gtaaccagtccttttatcgacaggctaggccg

SEQ ID NO: 6 - DNA, 148 bp, unidentified
  source 1..148: mol_type = other DNA
tttgaccctttatttgaccctctttttttggcatgtaaaaaaatcctttaaaatcaataggttaaaaataggctctatttttagggttatttggctatttttgcccgatattcctttcatttagggggatttttaattatttactcta

SEQ ID NO: 7 - DNA, 28 bp, unidentified
  source 1..28: mol_type = other DNA
gttcactgccgcacaggcagcttagaaa

SEQ ID NO: 8 - DNA, 32 bp, unidentified
  source 1..32: mol_type = other DNA
tgcgttttgaactgattccgcagggtaaaacc

SEQ ID NO: 9 - DNA, 36 bp, unidentified
  source 1..36: mol_type = other DNA
gaaagtaaccagtccttttatcgacaggctaggccg

SEQ ID NO: 10 - DNA, 36 bp, unidentified
  source 1..36: mol_type = other DNA
gaaccggcctagcctgtcgataaaaggactggttac

SEQ ID NO: 11 - DNA, 36 bp, unidentified
  source 1..36: mol_type = other DNA
taagggattttggtcatgagcccttttgcgccagcc

SEQ ID NO: 12 - DNA, 46 bp, unidentified
  source 1..46: mol_type = other DNA
agctgtcaaacatgagaattcatactctctatgtttccaatggagt

SEQ ID NO: 13 - DNA, 39 bp, unidentified
  source 1..39: mol_type = other DNA
cagggcggggcgtaaaggttagtttttggaactacacct

SEQ ID NO: 14 - DNA, 44 bp, unidentified
  source 1..44: mol_type = other DNA
tcacatgttctttcctgcgtgaattgaaataactgatggcaagc

SEQ ID NO: 15 - DNA, 26 bp, unidentified
  source 1..26: mol_type = other DNA
aattctcatgtttgacagcttatcat

SEQ ID NO: 16 - DNA, 17 bp, unidentified
  source 1..17: mol_type = other DNA
ttacgccccgccctgcc

SEQ ID NO: 17 - DNA, 22 bp, unidentified
  source 1..22: mol_type = other DNA
acgcaggaaagaacatgtgagc

SEQ ID NO: 18 - DNA, 25 bp, unidentified
  source 1..25: mol_type = other DNA
ctcatgaccaaaatcccttaacgtg

SEQ ID NO: 19 - DNA, 36 bp, unidentified
  source 1..36: mol_type = other DNA
gaaatgcgttttgaactgattccgcagggtaaaacc

SEQ ID NO: 20 - DNA, 36 bp, unidentified
  source 1..36: mol_type = other DNA
gaacggttttaccctgcggaatcagttcaaaacgca

SEQ ID NO: 21 - DNA, 40 bp, unidentified
  source 1..40: mol_type = other DNA
ggtcaccagctcaccgtctgttaggcgagaagggaaaggg

SEQ ID NO: 22 - DNA, 55 bp, unidentified
  source 1..55: mol_type = other DNA
gttgggttgagccgcgatagtcgttaaatattcagatagacggagataataaacg

SEQ ID NO: 23 - DNA, 16 bp, unidentified
  source 1..16: mol_type = other DNA
tcacgcccgacgccag

SEQ ID NO: 24 - DNA, 41 bp, unidentified
  source 1..41: mol_type = other DNA
gctcgagatctgatatcactcaccctctggtgattgtcgat

SEQ ID NO: 25 - DNA, 18 bp, unidentified
  source 1..18: mol_type = other DNA
ctatcgcggctcaaccca

SEQ ID NO: 26 - DNA, 46 bp, unidentified
  source 1..46: mol_type = other DNA
tggcgtcgggcgtgaaacaaaatctaggtttctgacgttaataaag

SEQ ID NO: 27 - DNA, 31 bp, unidentified
  source 1..31: mol_type = other DNA
agtgatatcagatctcgagctcggtacccgg

SEQ ID NO: 28 - DNA, 20 bp, unidentified
  source 1..20: mol_type = other DNA
agacggtgagctggtgacct

SEQ ID NO: 29 - DNA, 34 bp, unidentified
  source 1..34: mol_type = other DNA
cgaagcgcgagttccccatagatctcgagctcgg

SEQ ID NO: 30 - DNA, 39 bp, unidentified
  source 1..39: mol_type = other DNA
ggaactcgcgcttcggacgctcgagagatctgatatcac

SEQ ID NO: 31 - DNA, 20 bp, unidentified
  source 1..20: mol_type = other DNA
ttcctgtcggtgtgcttgaa

SEQ ID NO: 32 - DNA, 20 bp, unidentified
  source 1..20: mol_type = other DNA
cggacagcacagcatcattc

SEQ ID NO: 33 - DNA, 24 bp, unidentified
  source 1..24: mol_type = other DNA
agcttgatccatcacccatattac

SEQ ID NO: 34 - DNA, 17 bp, unidentified
  source 1..17: mol_type = other DNA
tctaccattgcggcgtc

SEQ ID NO: 35 - DNA, 24 bp, unidentified
  source 1..24: mol_type = other DNA
tcgcatctgccttgtttgtataag

SEQ ID NO: 36 - DNA, 25 bp, unidentified
  source 1..25: mol_type = other DNA
atgaatactgcgcagaatcagactc

SEQ ID NO: 37 - DNA, 20 bp, unidentified
  source 1..20: mol_type = other DNA
attactgaggaaccgcctcg

SEQ ID NO: 38 - DNA, 20 bp, unidentified
  source 1..20: mol_type = other DNA
tgcgattccaggatatacgg

SEQ ID NO: 39 - DNA, 26 bp, unidentified
  source 1..26: mol_type = other DNA
aggatggtcgatcttcagctattgtg

SEQ ID NO: 40 - DNA, 20 bp, unidentified
  source 1..20: mol_type = other DNA
gtgaaccgccaaaaactcgg

SEQ ID NO: 41 - DNA, 20 bp, unidentified
  source 1..20: mol_type = other DNA
atcatcctcccgtcattgcc